(19) United States
(12) Patent Application Publication
(10) Pub. No.: US 2011/0250646 A1
(43) Pub. Date: Oct. 13, 2011

(54) AMMONIA PRETREATMENT OF BIOMASS FOR IMPROVED INHIBITOR PROFILE

(75) Inventors: STEPHANE FRANCOIS BAZZANA, WILMINGTON, DE (US); CARL E. CAMP, WILMINGTON, DE (US); BRADLEY CURT FOX, BEAR, DE (US); RINALDO SORIA SCHIFFINO, WILMINGTON, DE (US); KEITH DUMONT WING, WILMINGTON, DE (US); YAMAIRA GONZALEZ, WILMINGTON, DE (US)

(73) Assignee: E.I. DU PONT DE NEMOURS AND COMPANY, WILMINGTON, DE (US)

(21) Appl. No.: 12/900,586

(22) Filed: Oct. 8, 2010

Related U.S. Application Data
(60) Provisional application No. 61/250,598, filed on Oct. 12, 2009.

(51) Int. Cl. C12P 19/00; C12P 7/14; C12P 7/18; C12P 7/16

(52) U.S. Cl. 435/72; 435/160; 435/162; 435/158

(57) ABSTRACT

Methods for treating biomass for release of fermentable sugars with an improved inhibitor profile are provided. Specifically, a hydrolysate comprising fermentable sugars with an improved inhibitor profile is obtained by saccharification of a reaction product obtained by pretreating biomass with ammonia under suitable reaction conditions. The pretreated biomass reaction product has an acetamide to acetate molar ratio greater than about 1 and an acetyl conversion of greater than 60%. The acetamide to acetate molar ratio is maintained greater than about 1 throughout saccharification. The hydrolysate may be fermented to a target compound.

### CROSS-REFERENCE TO RELATED APPLICATION

This application claims the benefit of priority of U.S. Provisional Application No. 61/250,598 filed on Oct. 12, 2009, which is herein incorporated by reference in its entirety.

### FIELD OF THE INVENTION

Methods for treating biomass to obtain fermentable sugars are provided. Specifically, methods for treating biomass with ammonia for release of fermentable sugars with an improved inhibitor profile are described. Also described are improved methods for fermenting sugars to a target product.

### BACKGROUND OF THE INVENTION

Production of ethanol by microorganisms provides an alternative energy source to fossil fuels and is an important area of current research. Cellulosic hydrolysates are desired as renewable sources of sugars for fermentation media for production of ethanol by microorganisms. Cellulosic hydrolysates are generally produced from biomass by pretreatment and saccharification. Various pretreatment methods are known, including ammonia pretreatment of biomass.

For example, methods for ammoniation of straw and other plant materials containing lignocellulose with a dry matter content of at least 60% is disclosed in U.S. Pat. No. 4,064,276. Anhydrous ammonia is used and the ammonia impregnated material is left at ambient temperature for at least 10 days.

U.S. Pat. No. 5,037,663 discloses a process for treating cellulose and/or hemicellulose containing feedstuff materials with liquid ammonia in which the weight ratio of ammonia to dry fiber can vary from about 0.5 to about 10 parts ammonia to about 1 part material. In general the optimum moisture content will be from about 10 to about 40% total moisture on a dry basis and treatment pressures from about 150 to about 500 psi can be employed. After treatment the pressure is then rapidly reduced to atmospheric.

Published Patent Application US 2007/0031918 discloses methods for pretreating biomass under conditions of high solids and low ammonia concentration. The concentration of ammonia used is minimally a concentration that is sufficient to maintain the pH of the biomass-aqueous ammonia mixture alkaline and maximally less than about 12 weight percent relative to dry weight of biomass. The dry weight of biomass is at an initial concentration of at least about 15% up to about 80% of the weight of the biomass-aqueous ammonia mixture.

Published Patent Application US 2008/0008783 discloses a process for the treatment of a plant biomass to increase the reactivity of plant polymers, comprising hemicellulose and cellulose, which comprises: contacting the plant biomass, which has been ground and which contains varying moisture contents, with anhydrous ammonia in the liquid or vapor state, and/or concentrated ammonia:water mixtures in the liquid or vapor state, to obtain a mixture in which the ratio of ammonia to dry biomass is between about 0.2 to 1 and 1.2 to 1, and the water to dry biomass ratio is between about 0.2 to 1.0 and 1.5 to 1. The temperature is maintained between about 50° C. and 140° C. and the pressure is rapidly released by releasing ammonia from the vessel to form a treated biomass.

Cellulosic hydrolysates typically contain substances that can be detrimental to biocatalyst growth and production. For example, acetate is a common product present in cellulosic hydrolysates which has been shown to be inhibitory to Zymomonas mobilis at concentrations routinely found in hydrolysate (Ranatunga et al. (1997) Applied Biochemistry and Biotechnology 67:185-198).

Methods for pretreating biomass with ammonia which provide hydrolysates after saccharification having an improved inhibitor profile are desired. Hydrolysates with improved inhibitor profiles would be advantageous for use in fermentation of sugars to target products and could provide economic benefits.

### SUMMARY OF THE INVENTION

The present invention provides methods for treating biomass for release of fermentable sugars with an improved inhibitor profile. The methods include treating biomass with an amount of ammonia under suitable reaction conditions to provide for a pretreated biomass reaction product having an acetamide to acetate molar ratio greater than about 1 and an acetyl conversion of greater than 60%, and saccharifying the reaction product with at least one saccharification enzyme while maintaining the acetamide to acetate molar ratio greater than about 1 throughout the saccharifying step. Suitable reaction conditions include pressure from about sub-atmospheric pressure to less than 10 atmospheres, a mass ratio of water to ammonia of less than about 20:1, a temperature of about 4° C. to about 200° C., a reaction time of 30 days or less, and a system solids loading of greater than about 60%.

In one embodiment of the invention, a method is provided, the method comprising:

a) treating biomass with an amount of ammonia under suitable reaction conditions wherein said conditions provide for a pretreated biomass reaction product having an acetamide to acetate molar ratio greater than about 1 and an acetyl conversion of greater than 60%, wherein said suitable reaction conditions include pressure from about sub-atmospheric pressure to less than 10 atmospheres;

b) saccharifying the pretreated biomass reaction product with an enzyme consortium, wherein a hydrolysate comprising fermentable sugars is produced and wherein said hydrolysate has an improved inhibitor profile compared to saccharifying a pretreated biomass reaction product having an acetamide to acetate molar ratio of less than about 1; and

c) maintaining the acetamide to acetate molar ratio greater than about 1 throughout the saccharifying of step (b).

In some embodiments, the method may further comprise fermenting the hydrolysate to produce a target product by adding an inoculum of seed cells capable of fermenting sugars to a target product. In some embodiments, the acetyl conversion is greater than 70%. In some embodiments, the total xylose yield through saccharification is improved compared to that for a pretreated biomass reaction product having an acetamide to acetate molar ratio of less than about 1 and an acetyl conversion of greater than 60%. In some embodiments, the biomass has a dry matter content of at least about 60 weight percent in step (a). In some embodiments, the biomass is subjected to preprocessing prior to step (a). In some embodiments, the temperature is about 20° C. to about 121° C. and the reaction time is about 100 hours or less.

The present invention provides methods for fermenting sugars to a target product. The methods include providing a hydrolysate having an acetamide to acetate molar ratio greater than about 1, adding an inoculum of appropriate seed cells to the hydrolysate, and fermenting the hydrolysate to provide a fermentation mixture comprising a target product. The hydrolysate may be obtained by saccharifying a pre-treated biomass reaction product as described above. In one embodiment of the invention, an improved method for fermenting sugars to a target product is provided, the method comprising:

a) providing a hydrolysate having an acetamide to acetate molar ratio of greater than about 1;

b) adding an inoculum of seed cells capable of fermenting sugars to a target product to the hydrolysate, wherein the inoculum is about 0.1 percent to about 10 percent of the hydrolysate; and

c) fermenting the hydrolysate to provide a fermentation mixture comprising a target product.

In some embodiments, the hydrolysate provides for improved cell growth rate of said inoculum compared to a hydrolysate having an acetamide to acetate molar ratio of less than 1. In some embodiments, fermenting the hydrolysate is initiated with a lower inoculum of seed cells compared to fermenting a hydrolysate having an acetamide to acetate molar ratio of less than 1. In some embodiments, the target product is selected from the group consisting of ethanol, butanol, and 1,3-propanediol.

### DETAILED DESCRIPTION OF THE INVENTION

The present invention provides a method for treating biomass for release of fermentable sugars with an improved inhibitor profile. The methods include treating biomass with an amount of ammonia under suitable reaction conditions to provide for a pretreated biomass reaction product having an acetamide to acetate molar ratio greater than about 1 and an acetyl conversion of greater than 60%, and saccharifying the reaction product with an enzyme consortium while maintaining the acetamide to acetate molar ratio greater than about 1 throughout saccharification.

In addition, the present invention provides a method for fermenting sugars to a target product. The methods include providing a hydrolysate having an acetamide to acetate molar ratio of greater than about 1, adding an inoculum of seed cells to the hydrolysate, and fermenting the hydrolysate to provide a fermentation mixture comprising the target product. The hydrolysate is produced by saccharification of a pre-treated biomass, with the acetamide to acetate molar ratio being maintained through the saccharification. The inoculum is about 0.1 percent to about 10 percent of the hydrolysate.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### DEFINITIONS

The following definitions are used in this disclosure:

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

“Room temperature” and “ambient” when used in reference to temperature refer to any temperature from about 15° C. to about 25° C.

“Fermentable sugars” refers to a sugars comprising monosaccharides and polysaccharides that can be used as a carbon source by a microorganism in a fermentation process to produce a target product.

“Monomeric sugars” or “simple sugars” consist of a single pentose or hexose unit, e.g., glucose.

“Lignocellulosic” refers to material comprising both lignin and cellulose. Lignocellulosic material may also comprise hemicellulose.

“Cellulosic” refers to a composition comprising cellulose.

“Acetyl conversion” refers to the hydrolysis or ammonolysis of biomass acetyl ester groups to produce acetic acid in equilibrium with ammonium acetate (from hydrolysis) or acetamide (from ammonolysis).

“Target product” refers to a chemical, fuel, or chemical building block produced by fermentation. Product is used in a broad sense and includes molecules such as proteins, including, for example, peptides, enzymes, and antibodies. Also contemplated within the definition of target product are ethanol and butanol.

“Dry weight of biomass” refers to the weight of the biomass having all or essentially all water removed. Dry weight is typically measured according to American Society for Testing and Materials (ASTM) Standard E1756-01 (Standard Test Method for Determination of Total Solids in Biomass) or Technical Association of the Pulp and Paper Industry, Inc. (TAPPI) Standard T-412 om-02 (Moisture in Pulp, Paper and Paperboard). Dry weight of biomass is synonymous with dry matter content of biomass.

“System solids loading” refers to the dry weight of biomass within the system divided by the total system mass, which includes water, ammonia, and biomass, including other additives to the pretreatment process.

“Biomass” and “lignocellulosic biomass” as used herein refer to any lignocellulosic material, including cellulosic and hemi-cellulosic material, for example, bioenergy crops, agricultural residues, municipal solid waste, industrial solid waste, yard waste, wood, forestry waste, and combinations thereof, and as further described below. Biomass has a carbohydrate content that comprises polysaccharides and oligosaccharides and may also comprise additional components, such as protein and/or lipid.

“Improved inhibitor profile” means an inhibitor profile which has decreased levels of any known fermentation inhibitors. Examples of fermentation inhibitors are acetate and acetic acid.

“Cell growth rate” means the maximum exponential growth rate of a microorganism during fermentation. This is measured as a slope of a line passing through a plot of the In(OD) of a growing culture of a microorganism vs time and has units of hr⁻¹.

“OD” is the measured optical density of a culture of a microorganism and is proportional to the total number of cells per unit volume.

“Initial growth lag” means the time required for a growing culture of a microorganism to reach its maximum exponential growth rate after inoculation into a growth medium. This is measured at the intercept between the line measuring the slope of a plot of the In(OD) versus time and a horizontal line at the value of In(OD) at time zero.

“Preprocessing” as used herein refers to processing of lignocellulosic biomass prior to pretreatment. Preprocessing is any treatment of biomass that prepares the biomass for pretreatment, such as mechanically chopping and/or drying to the appropriate moisture contact.

“Saccharification” and “saccharifying” refer to the production of fermentable sugars from polysaccharides by the action of acids, bases, or hydrolytic enzymes. Production of fermentable sugars from pretreated biomass occurs by enzymatic saccharification by the action of cellulolytic and hemicellulolytic enzymes.

“Pretreating biomass” or “biomass pretreatment” as used herein refers to subjecting native or preprocessed biomass to chemical, physical, or biological action, or any combination thereof, rendering the biomass more susceptible to enzymatic saccharification or other means of hydrolysis prior to saccharification. For example, the methods claimed herein may be referred to as pretreatment processes that contribute to rendering biomass more accessible to hydrolytic enzymes for saccharification.

“Pretreated biomass” as used herein refers to native or preprocessed biomass that has been subjected to chemical, physical, or biological action, or any combination thereof, rendering the biomass more susceptible to enzymatic saccharification or other means of hydrolysis prior to saccharification.

“Hydrolysate” refers to the liquid in contact with the lignocellulosic biomass which contains the products of hydrolytic reactions acting upon the biomass (either enzymatic or not), in this case monomeric and oligomeric sugars.

“Enzyme consortium” or “saccharification enzyme consortium” as used herein refers to a collection of enzymes, usually secreted by microorganisms, which in the present case will typically contain one or more cellulases, xylanases, glycosidases, ligninases and feruloyl esterases.

“Titer” refers to the total amount of target product per unit volume of the medium, for example ethanol, produced by fermentation per liter of fermentation medium.

### Lignocellulosic Biomass:

The lignocellulosic biomass pretreated herein includes, but is not limited to, bioenergy crops, agricultural residues, municipal solid waste, industrial solid waste, sludge from paper manufacture, yard waste, wood and forestry waste. Examples of biomass include, but are not limited to corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

In one embodiment, biomass that is useful for the invention includes biomass that has a relatively high carbohydrate content, is relatively dense, and/or is relatively easy to collect, transport, store and/or handle.

In one embodiment of the invention, biomass that is useful includes corn cobs, corn stover, sugar cane bagasse and switchgrass.

In another embodiment, the lignocellulosic biomass includes agricultural residues such as corn stover, wheat straw, barley straw, oat straw, rice straw, canola straw, and soybean stover; grasses such as switch grass, miscanthus, cord grass, and reed canary grass; fiber process residues such as corn fiber, beet pulp, pulp mill fines and rejects and sugar cane bagasse; sorghum; forestry wastes such as aspen wood, other hardwoods, softwood and sawdust; and post-consumer waste paper products; as well as other crops or sufficiently abundant lignocellulosic material.

The lignocellulosic biomass may be derived from a single source, or biomass can comprise a mixture derived from more than one source; for example, biomass could comprise a mixture of corn cobs and corn stover, or a mixture of stems or stalks and leaves.

The biomass may be used directly as obtained from the source, or may be subjected to some preprocessing, for example, energy may be applied to the biomass to reduce the size, increase the exposed surface area, and/or increase the accessibility of lignin and of cellulose, hemicellulose, and/or oligosaccharides present in the biomass to the ammonia treatment and to saccharification enzymes. Pre-processing means useful for reducing the size, increasing the exposed surface area, and/or increasing the accessibility of the lignin, and the cellulose, hemicellulose, and/or oligosaccharides present in the biomass to the ammonia treatment and to saccharification enzymes include, but are not limited to, milling, crushing, grinding, shredding, chopping, disc refining, ultrasound, and microwave. This application of energy may occur before or during the ammonia treatment step, before or during saccharification, or any combination thereof.

In one embodiment of the invention, prior to treatment with ammonia, the biomass has a dry matter content of at least about 60 weight percent, for example at least about 65, or at least about 70, or at least about 75, or at least about 80, or at least about 85, or at least about 90 weight percent dry matter. If necessary, drying biomass prior to pretreatment may occur by conventional means, such as by using rotary dryers, flash dryers, or superheated steam dryers.

### Ammonia:

As used herein, “ammonia” refers to the use of anhydrous ammonia gas (NH₃), ammonia gas in an aqueous medium, compounds comprising ammonium ions (NH₄⁺) such as ammonium hydroxide or ammonium sulfate, compounds that release ammonia upon degradation such as urea, and combinations thereof, optionally in an aqueous medium.

The amount of ammonia used in the present method is greater than the amount of acetyl ester groups contained in the biomass on a molar basis. For example, the amount of ammonia may be greater than about 3, 5, 10, 15, or 20 weight percent relative to dry weight of biomass. Depending on the biomass used, the amount of ammonia can be four to six times (on a weight basis) the amount of acetyl groups contained in the biomass.

Ammonia as used in the present process provides advantages over other bases. Ammonia partitions into a liquid phase and vapor phase. Gaseous ammonia can diffuse more easily through biomass than a liquid base, resulting in more efficacious pretreatment at lower concentrations. The use of ammonia also reduces the requirement to supplement growth medium used during fermentation with a nitrogen source. In addition, ammonia is a low-cost material and thus provides an economical process. Ammonia can also be recycled to the pretreatment reactor during pretreatment or following pretreatment, thus enabling a more economical process. For example, following pretreatment, as the temperature is decreased to that suitable for saccharification, ammonia gas may be released, optionally in the presence of a vacuum, and may be recycled. In a continuous process, ammonia may be continuously recycled.

### Ammonia Treatment Conditions:

Pretreatment of biomass with ammonia can be carried out in any suitable vessel. Typically the vessel is one that can withstand pressure, has a mechanism for heating, and has a mechanism for mixing the contents. Commercially available vessels include, for example, the ZIPPERCLAVE® reactor (Autoclave Engineers, Erie, PA), the Jaygo reactor (Jaygo Manufacturing, Inc., Mahwah, N.J.), and a steam gun reactor ((described in General Methods Autoclave Engineers, Erie, PA). Much larger scale reactors with similar capabilities may be used. Alternatively, the biomass and ammonia may be combined in one vessel, then transferred to another reactor. In addition, biomass may be pretreated in one vessel, then further processed in another reactor such as a steam gun reactor (described in General Methods; Autoclave Engineers, Erie, PA).

The ammonia treatment may be performed in any suitable vessel, such as a batch reactor or a continuous reactor. The suitable vessel may be equipped with a means, such as impellers, for agitating the biomass-aqueous ammonia mixture. Reactor design is discussed in Lin, K.-H., and Van Ness, H. C. (in Perry, R. H. and Chilton, C. H. (eds), Chemical Engineer's Handbook, 5^{th} Edition (1973) Chapter 4, McGraw-Hill, NY). The ammonia treatment may be carried out as a batch process, or as a continuous process.

The ammonia treatment may be performed in a reactor system with or without mixing.

Prior to contacting the biomass with ammonia, vacuum may be applied to the vessel containing the biomass. By evacuating air from the pores of the biomass, better penetration of the ammonia into the biomass may be achieved. The time period for applying vacuum and the amount of negative pressure that is applied to the biomass will depend on the type of biomass and can be determined empirically so as to achieve optimal pretreatment of the biomass (as measured by the production of fermentable sugars following saccharification).

The treatment of biomass with ammonia may be carried out at pressures less than 10 atmospheres. For example, suitable reaction conditions can include pressure less than 9 atmospheres, or less than 8 atmospheres, or less than 7 atmospheres, or less than 6 atmospheres, or less than 5 atmospheres, or less than 4 atmospheres, or less than 3 atmospheres, or less than 2 atmospheres. Ammonia treatment may also be carried out at less than atmospheric pressure, provided that sufficient ammonia is used relative to the biomass for effective pretreatment to occur.

According to the present method, the treatment of biomass with ammonia is carried out under suitable reactions conditions including a temperature of about 4° C. to about 200° C. In another embodiment, treatment of biomass with ammonia is carried out at a temperature of from about 4° C. to about 150° C. In another embodiment, treatment of biomass with ammonia is carried out at a temperature of from about 4° C. to about 121° C. In another embodiment, treatment of biomass with ammonia is carried out at a temperature of from about 10° C. to about 100° C. In another embodiment, treatment of biomass with ammonia is carried out at a temperature of from about 20° C. to about 50° C.

The treatment of biomass with ammonia is carried out for a time period from about 20 minutes to about 200 hours. Longer periods of pretreatment, such as 30 days or several months, are possible, however a shorter period of time may be preferable for practical, economic reasons. Longer periods may provide the benefit of reducing the need for application of energy for breaking up the biomass, therefore, a period of time up to about 200 hours may be preferable.

In one embodiment, the ammonia treatment may be performed at a relatively high temperature for a relatively short period of time, for example at from about 140° C. to about 160° C. for about 20 minutes to about 30 minutes. In another embodiment, the ammonia treatment may be performed at a lower temperature for a relatively long period of time, for example from about 50° C. to about 100° C. for about 24 hours to about 48 hours. In one embodiment, the ammonia treatment may be performed at about 20° C. to about 121° C. for a reaction time of about 100 hours or less. In still another embodiment, the ammonia treatment may be performed at room temperature (approximately 22-26° C.) for an even longer period of time of about 30 days, or longer. Other temperature and time combinations intermediate to these may also be used.

According to the present method, suitable reaction conditions include a mass ratio of water to ammonia of less than about 20:1, for example of less than about 18:1, 16:1, 14:1, 12:1, 10:1, 8:1, 6:1, 4:1, 3:1, 2:1, 1:1, or about 0.5:1. In some embodiments, the mass ratio of water to ammonia can be below 0.5:1. Optionally, water in addition to the moisture contained in the biomass may be added to the biomass. During the ammonia treatment step, the water may be present as liquid water, gaseous water (water vapor), steam, or a combination thereof, and may be added to the biomass as liquid water, gaseous water, steam, or a combination thereof. The water may be added in combination with the ammonia, or the water and ammonia may be added separately. The water may be added concurrently with the ammonia, or before or after the ammonia addition.

According to the present method, suitable reaction conditions include a system solids loading of greater than about 60%, for example about 70%, about 80% or higher.

For the ammonia treatment step, the pressure, temperature, time for treatment, ammonia amount, mass ratio of water to ammonia, biomass type, biomass dry matter content, and biomass particle size are related; thus these variables may be adjusted as necessary to obtain an optimal product to be contacted with a saccharification enzyme consortium.

In order to obtain sufficient quantities of sugars from biomass, the biomass may be treated with ammonia one time or more than one time. Likewise, a saccharification reaction can be performed one or more times. Both ammonia treatment and saccharification processes may be repeated if desired to obtain higher yields of sugars. To assess performance of the ammonia treatment and saccharification processes, separately or together, the theoretical yield of sugars derivable from the starting biomass can be determined and compared to measured yields.

### Acetamide/Acetate Ratio and Acetyl Conversion:

Acetyl esters in lignocellulosic biomass can react with water to form acetic acid. In an aqueous ammonia system, the acetic acid will be in equilibrium with ammonium acetate. Ammonia is known to compete with hydrolysis, via ammonolysis, of acetyl esters in biomass to form acetamide. Acetamide is less toxic than acetate to certain fermentation organisms, such as Zymomonas mobilis as demonstrated for example in published U.S. patent application 2007/0031918. Thus conversion of acetyl esters to acetamide rather than to acetic acid reduces the need to remove acetic acid from the pretreated biomass reaction product or saccharification product.

A high molar extent of deacetylation of biomass, for example greater than about 60%, is desirable as this enables high xylose (monomer and oligomer) yields from xylan contained in the biomass. Since the cost of manufacturing is highly sensitive to the biomass cost, the cost is highly sensitive to sugar yield. Also, high sugar yield can result in higher ethanol concentrations in fermentation, which can reduce the downstream product recovery cost as well.

Another consideration is the acetic acid concentration in fermentation. Above about 5 g/L, acetic acid begins to slow down the growth rate of Zymomonas mobilis. A higher growth rate due to reduced inhibitor concentration (for example acetic acid or acetate), enables a lower volume of seed inoculum to be used, reducing the seed fermentor cost. Also, a higher growth rate can reduce the production scale fermentor cost.

### Saccharification:

Following treatment with ammonia, the pretreated biomass reaction product comprises a mixture of cellulose, hemicellulose, polysaccharides, lignin, remains of the other components of the biomass, and products of reaction of ammonia with the components of the biomass, specifically acetamide, acetic acid, and ammonium acetate. The ammonia-treated biomass reaction product has an acetamide to acetate molar ratio greater than about 1 and an acetyl conversion of greater than 60%, for example greater than about 65%, or greater than about 70%. As filtration and washing steps are not necessary to obtain improved sugar yields, and as the costs associated with them negatively impact the economics of the method, filtering and washing of the biomass is preferably omitted. The ammonia-treated biomass may be dried at room temperature. The concentration of glucan, xylan and lignin content of the ammonia-treated biomass may be determined using analytical means well known in the art.

The ammonia-treated biomass may then be further hydrolyzed or saccharified in the presence of at least one saccharification enzyme or an enzyme consortium to release oligosaccharides and/or monosaccharides in a hydrolysate. Surfactants such as polyethylene glycols (PEG) may be added to improve the saccharification process (U.S. Pat. No. 7,354,743 B2, incorporated herein by reference). Saccharification enzymes and methods for biomass treatment are reviewed in Lynd, L. R., et al. (Microbiol. Mol. Biol. Rev., 66:506-577, 2002). The saccharification enzyme consortium may comprise one or more glycosidases; the glycosidases may be selected from the group consisting of cellulose-hydrolyzing glycosidases, hemicellulose-hydrolyzing glycosidases, and starch-hydrolyzing glycosidases. Other enzymes in the saccharification enzyme consortium may include peptidases, lipases, ligninases and feruloyl esterases.

Saccharifying with an enzyme consortium comprises contacting biomass, or a pretreated biomass reaction product with one or more enzymes selected primarily, but not exclusively, from the group “glycosidases” which hydrolyze the ether linkages of di-, oligo-, and polysaccharides and are found in the enzyme classification EC 3.2.1.x (Enzyme Nomenclature 1992, Academic Press, San Diego, Calif. with Supplement 1 (1993), Supplement 2 (1994), Supplement 3 (1995, Supplement 4 (1997) and Supplement 5 [in Eur. J. Biochem., 223:1-5, 1994; Eur. J. Biochem., 232:1-6, 1995; Eur. J. Biochem., 237:1-5, 1996; Eur. J. Biochem., 250:1-6, 1997; and Eur. J. Biochem., 264:610-650 1999, respectively]) of the general group “hydrolases” (EC 3.). Glycosidases useful in the present method can be categorized by the biomass component that they hydrolyze. Glycosidases useful for the present method include cellulose-hydrolyzing glycosidases (for example, cellulases, endoglucanases, exoglucanases, cellobiohydrolases, β-glucosidases), hemicellulose-hydrolyzing glycosidases (for example, xylanases, endoxylanases, exoxylanases, β-xylosidases, arabino-xylanases, mannases, galactases, pectinases, glucuronidases), and starch-hydrolyzing glycosidases (for example, amylases, α-amylases, β-amylases, glucoamylases, α-glucosidases, isoamylases). In addition, it may be useful to add other activities to the saccharification enzyme consortium such as peptidases (EC 3.4.x.y), lipases (EC 3.1.1.x and 3.1.4.x), ligninases (EC 1.11.1.x), and feruloyl esterases (EC 3.1.1.73) to help release polysaccharides from other components of the biomass. It is well known in the art that microorganisms that produce polysaccharide-hydrolyzing enzymes often exhibit an activity, such as cellulose degradation, that is catalyzed by several enzymes or a group of enzymes having different substrate specificities. Thus, a “cellulase” from a microorganism may comprise a group of enzymes, all of which may contribute to the cellulose-degrading activity. Commercial or non-commercial enzyme preparations, such as cellulase, may comprise numerous enzymes depending on the purification scheme utilized to obtain the enzyme. Thus, the saccharification enzyme consortium of the present method may comprise enzyme activity, such as “cellulase”, however it is recognized that this activity may be catalyzed by more than one enzyme.

Saccharification enzymes may be obtained commercially, in isolated form, such as SPEZYME® CP cellulase (Genencor International, Rochester, N.Y.) and MULTIFECT® xylanase (Genencor). In addition, saccharification enzymes may be expressed in host organisms at the biofuels plant, including using recombinant microorganisms.

One skilled in the art would know how to determine the effective amount of enzymes to use in the consortium and adjust conditions for optimal enzyme activity. One skilled in the art would also know how to optimize the classes of enzyme activities required within the consortium to obtain optimal saccharification of a given pretreatment product under the selected conditions.

Preferably the saccharification reaction is performed at or near the temperature and pH optima for the saccharification enzymes. The temperature optimum used with the saccharification enzyme consortium in the present method ranges from about 15° C. to about 100° C. In another embodiment, the temperature optimum ranges from about 20° C. to about 80° C. and most typically 45-50° C. The pH optimum can range from about 2 to about 11. In another embodiment, the pH optimum used with the saccharification enzyme consortium in the present method ranges from about 4 to about 6.5.

The saccharifying can be performed for a time of about several minutes to about 200 hours, and preferably from about 24 hours to about 72 hours. The time for the reaction will depend on enzyme concentration and specific activity, as well as the substrate used and the environmental conditions, such as temperature and pH. One skilled in the art can readily determine optimal conditions of temperature, pH and time to be used with a particular substrate and saccharification enzyme(s) consortium.

The saccharifying can be performed batch-wise, fed-batch or as a continuous process. The saccharification can also be performed in one step, or in a number of steps. For example, different enzymes required for saccharification may exhibit different pH or temperature optima. A primary treatment can be performed with enzyme(s) at one temperature and pH, followed by secondary or tertiary (or more) treatments with different enzyme(s) at different temperatures and/or pH. In addition, treatment with different enzymes in sequential steps may be at the same pH and/or temperature, or different pHs and temperatures, such as using hemicellulases stable and more active at higher pHs and temperatures followed by cellulases that are active at lower pHs and temperatures.

The acetamide to acetate molar ratio remains unchanged and/or is maintained through saccharification as long as the temperature and pH are maintained within the ranges described above.

The degree of solubilization of sugars from biomass following saccharification can be monitored by measuring the release of monosaccharides and oligosaccharides. Methods to measure monosaccharides and oligosaccharides are well known in the art. For example, the concentration of reducing sugars can be determined using the 1,3-dinitrosalicylic (DNS) acid assay (Miller, G. L., Anal. Chem., 31: 426-428, 1959). Alternatively, sugars can be measured by HPLC using an appropriate column as described below.

### Further Processing:

Fermentation to Target Products:

The hydrolysate comprising fermentable sugars and having an improved inhibitor profile produced by the present methods can be subjected to a fermenting step in which hydrolysate is contacted with an inoculum of seed cells capable of fermenting sugars to produce a fermentation mixture comprising one or more target products. “Fermentation” refers to any fermentation process or any process comprising a fermentation step. Target products include, without limitation alcohols (e.g., arabinitol, butanol, ethanol, glycerol, methanol, 1,3-propanediol, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, and xylonic acid); ketones (e.g., acetone); amino acids (e.g., aspartic acid, glutamic acid, glycine, lysine, serine, and threonine); gases (e.g., methane, hydrogen (H₂), carbon dioxide (CO₂), and carbon monoxide (CO)). The fermentation mixture may also include co-products, by-products, enzymes and other materials.

Fermentation processes also include processes used in the consumable alcohol industry (e.g., beer and wine), dairy industry (e.g., fermented dairy products), leather industry, and tobacco industry.

Further to the above, the sugars produced from saccharifying the pretreated biomass as described herein may be used to produce in general, organic products, chemicals, fuels, commodity and specialty chemicals such as xylose, acetone, acetate, glycine, lysine, organic acids (e.g., lactic acid), 1,3-propanediol, butanediol, glycerol, ethylene glycol, furfural, polyhydroxyalkanoates, cis, cis-muconic acid, and animal feed (Lynd, L. R., Wyman, C. E., and Gerngross, T. U., Biocommodity Engineering, Biotechnol. Prog., 15: 777-793, 1999; and Philippidis, G. P., Cellulose bioconversion technology, in Handbook on Bioethanol: Production and Utilization, Wyman, C. E., ed., Taylor & Francis, Washington, D.C., 179-212, 1996; and Ryu, D. D. Y., and Mandels, M., Cellulases: biosynthesis and applications, Enz. Microb. Technol., 2: 91-102, 1980).

Potential coproducts may also be produced, such as multiple organic products from fermentable carbohydrate. Lignin-rich residues remaining after pretreatment and fermentation can be converted to lignin-derived chemicals, chemical building blocks or used for power production.

Conventional methods of fermentation and/or saccharification are known in the art including, but not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC).

SHF uses separate process steps to first enzymatically hydrolyze cellulose to sugars such as glucose and xylose and then ferment the sugars to ethanol. In SSF, the enzymatic hydrolysis of cellulose and the fermentation of glucose to ethanol is combined in one step (Philippidis, G. P., in Handbook on Bioethanol: Production and Utilization, Wyman, C. E., ed., Taylor & Francis, Washington, D.C., 179-212, 1996). SSCF includes the cofermentation of multiple sugars (Sheehan, J., and Himmel, M., Bioethanol, Biotechnol. Prog. 15: 817-827, 1999). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., Microbiol. Mol. Biol. Reviews, 66: 506-577, 2002).

These processes may be used to produce target products from fermentation of the hydrolysates obtained by saccharification of biomass produced by the ammonia-treatment methods described herein. Target products produced in fermentation may be recovered using various methods known in the art. Products may be separated from other fermentation components by centrifugation, filtration, microfiltration, and nanofiltration. Products may be extracted by ion exchange, solvent extraction, or electrodialysis. Flocculating agents may be used to aid in product separation. As a specific example, bioproduced ethanol may be isolated from the fermentation medium using methods known in the art for ABE fermentations (see for example, Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. Then, the ethanol may be isolated from the fermentation medium using methods such as distillation, azeotropic distillation, liquid-liquid extraction, adsorption, gas stripping, membrane evaporation, or pervaporation.

### Advantages of the Present Methods:

It is well known that the hemicellulose component of biomass contains a significant amount of acetyl groups attached to the xylose units of the polymeric xylan. The acetyl groups block the action of the saccharification enzymes acting on hemicellulose and thus lower the yield of fermentable sugars. The acetyl esters must be removed to achieve maximum yields of fermentable sugars. However, the product acetic acid is a potent fermentation inhibitor when the fermentations are performed below pH 7. To achieve improved fermentation performance the acetic acid must either be removed or modified to a non-toxic chemical. The conversion of acetyl esters to acetamide by ammonolysis with ammonia in this pretreatment provides a process for conversion of the acetyl groups to a non-toxic chemical.

One of the advantages of the present methods is the improved yield of sugars which are obtained in saccharification of hydrolysates having an acetyl conversion of greater than 60%. In particular, the yield of xylose obtained through saccharification is improved with the present methods, which is of economic benefit.

Another advantage of the present methods is the improved rate of fermentation for hydrolysates having an acetamide to acetate molar ratio greater than about 1. Another advantage of the present methods is the possibility of running fermentations at lower pH, which can provide cost savings through lower usage of base to raise pH.

Additionally, hydrolysates having an acetamide to acetate molar ratio of greater than about 1 do not require as much inoculum for fermentation as do other hydrolysates. For example, with the present methods an inoculum of about 1.3% of the hydrolysate can be used in place of the typical inoculum amount of about 10% of the hydrolysate. A reduced inoculum enables the use of a smaller seed product tank, which is of economic benefit due to reduction of the costs associated with providing the inoculum.

A further advantage of the present methods is the potential to integrate ammonia treatment with biomass storage. For example, after harvest biomass may be treated with ammonia prior to storage in a silo, pile, or bunker system, which would also have the benefit of minimizing feedstock contamination due to mold or vermin. Alternatively, after harvest biomass may be treated with ammonia prior to storage in a biorefinery feedstock storage system, which would also have the benefit of reducing the capital cost associated with alternative pretreatment using high pressure, high temperature, and mechanically agitated reactors.

### EXAMPLES

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions.

The following abbreviations are used:

“HPLC” is High Performance Liquid Chromatography, “C” is Celsius, “kPa” is kiloPascal, “m” is meter, “mm” is millimeter, “μm” is micrometer, “μL” is microliter, “mL” is milliliter, “L” is liter, “N” is normal, “min” is minute, “mM” is millimolar, “cm” is centimeter, “g” is gram, “kg” is kilogram, “wt” is weight, “h” or “hr” is hour, “temp” or “T” is temperature, “theoret” is theoretical, “DM” is dry matter, “DWB” is dry weight of biomass, “ASME” is the American Society of Mechanical Engineers, “s.s.” is stainless steel, “in” or “ ”” is inch, “rpm” is revolutions per minute, “GUR” is glucose uptake rate, “XUR” is xylose uptake rate, “EtOH” is ethanol, “Max” is maximum, “Avg” is average, “Ex.” Is Example, “Comp.” is Comparative, and “OD” is optical density.

Sulfuric acid, ammonium hydroxide, acetic acid, acetamide, yeast extract, glucose, xylose, sorbitol, MgSO₄.7H₂O, phosphoric acid and citric acid were obtained commercially. Ammonium hydroxide solution was obtained from VWR (West Chester, Pa.). The enzyme cocktails were obtained from Genencor (Rochester, N.Y.) and from Novozyme (Salem, Va.). All commercial reagents were used as received unless otherwise specified.

Corn cob was obtained from University of Wisconsin Farm, in Madison, Wis. and was hammer milled to ⅜″ particles. The composition of the cob was determined by NREL biomass analysis procedures (Determination of Structural Carbohydrates and Lignin in Biomass) to be as follows:

TABLE 1
Composition of Corn Cob Used.

| | |
|---|---|
| Glucan | 34.78% |
| Xylan | 29.21% |
| Arabinan | 4.63% |
| Galactan | 1.43% |
| Mannan | 0.82% |
| Sucrose | 2.75% |
| Starch | 1.17% |
| Lignin | 12.80% |
| Acetyl | 2.47% |
| Protein | 0.76% |
| Ash | 1.00% |
| Uronic Acids | 3.94% |
| Water extractives | 3.15% |
| EtOH Extractives | 1.08% |
| total | 100.0% |

Dry matter content of biomass was determined using a Denver Instruments IR-120 moisture analyzer operating at 105° C.

To determine percent residual ammonia in treated biomass, approximately 15 g of treated biomass were mixed with deionized water to a total weight of approximately 100 g. The resulting slurry was mixed for 15 minutes at room temperature in a covered beaker. The water extract was separated from the bulk solids by decanting through a coarse filtration medium such as a Wipeall. Approximately 20 mL of the water extract were titrated to pH 5.0 using 0.1 N HCl. The titration was done using an autotitrator (Mettler, Toledo, Rondo 60). The equivalents of acid required to reach pH 5.0 were converted to equivalents of NH₃. Results were reported normalized to the amount of dry matter in the biomass sample before ammonia treatment.

### Measurement of Cellulose and Hemicellulose in Biomass

The composition of biomass is measured by any one of the standard methods well known in the art, such as ASTM E1758-01 “Standard method for the determination of carbohydrates by HPLC”. Measurement of sugars, acetamide, acetic acid, and lactic acid content

Soluble sugars (glucose, cellobiose, xylose, xylobiose, galactose, arabinose, and mannose) acetamide, acetic acid, and lactic acid in saccharification liquor were measured by HPLC (Agilent Model 1200, Agilent Technologies, Palo Alto, Calif.) using Bio-Rad HPX-87P and Bio-Rad HPX-87H columns (Bio-Rad Laboratories, Hercules, Calif.) with appropriate guard columns. Acetate in the samples was measured and reported as acetic acid. The HPLC run conditions were as follows:
Biorad Aminex HPX-87H (for carbohydrates, acetamide, acetic acid and lactic acid)
Injection volume: 5-10 μL, dependent on concentration and detector limits
Mobile phase: 0.01 N Sulfuric acid, 0.2 μm filtered and degassed
Flow rate: 0.6 mL/minute
Column temperature: 55° C.
Detector temperature: as close to column temperature as possible
Detector: refractive index
Run time: 25-75 minutes data collection

After the run, concentrations in the sample were determined from standard curves for each of the compounds.

Monosaccharides were directly measured in the hydrolysate. The insoluble matter was removed from the hydrolysate by centrifuge. The pH of the separated liquid was adjusted, if necessary, to 5-6 for Bio-Rad HPX-87P column and to 1-3 for Bio-Rad HPX-87H column, with sulfuric acid. The separated liquid was diluted, if necessary, then filtered by passing through a 0.2 micron syringe filter directly into an HPLC vial.

For analysis of total dissolved sugars, 10 mL of diluted sample was placed in a pressure vial and 349 μL of 75% H2SO4 was added. The vial was capped and placed in the autoclave for an hour to hydrolyze all sugars to monosaccharides. The samples were cooled and their pH was adjusted by sodium carbonate to the necessary pH, as described above, then the samples were filtered into the HPLC vials and analyzed by HPLC.

The HPLC run conditions were as follows:
Biorad Aminex HPX-87P (for carbohydrates):
Injection volume: 10-50 μL, dependent on concentration and detector limits
Mobile phase: HPLC grade water, 0.2 μm filtered and degassed
Flow rate: 0.6 mL/minute
Column temperature: 80-85° C., guard column temperature <60° C.
Detector temperature: as close to main column temperature as possible
Detector: refractive index
Run time: 35 minutes data collection plus 15 minutes post run (with possible adjustment for later eluting compounds)
   After the run, concentrations in the sample were determined from standard curves for each of the compounds.

Analyses of fermentation products were done with a Waters Alliance HPLC system. The column used was a Transgenomic ION-300 column (#ICE-99-9850, Transgenomic, Inc., Omaha, Nebr.) with a BioRad Micro-Guard Cartridge Cation-H (#125-0129, Bio-Rad, Hercules, Calif.). The column was run at 75° C. and 0.4 mL/min flow rate using 0.01 NH₂SO₄ as solvent. The concentrations of starting sugars and products were determined with a refractive index detector using external standard calibration curves.

### Ammonia Treatment Equipment

Ammonia treatment experiments were performed using two sets of equipment. One system consisted of a 5 L horizontal cylindrical pressure vessel (Littleford Day, Florence, Ky.) modified to include a 1.5″ ball valve on the top of the reactor, which could be removed to charge biomass. The reactor was equipped with two ports in the headspace, a 1.5″ ball valve on the bottom, various thermocouples, a relief valve, a pressure gage, and a pressure transducer. The reactor contained a so-called “heat transfer” type impeller, which contained four blades for mixing solids vertically and horizontally. The impeller was rotated at approximately 40 rpm for all experiments. A Cole-Palmer drive fitted with a gear pump head was used to meter water or aqueous ammonia solution into the reactor using a bottle placed on an electronic balance. A Teledyne ISCO high pressure syringe pump (model D500) retrofitted with a high temperature pressure transducer, and wrapped with an elastomer-encapsulated heat tape was used to pre-heat aqueous ammonia solutions. A needle valve connected to the top flange was used to control the pressure flash and vacuum flash. The flash vapors were passed through a tube-in-tube heat exchanger which used house cold water. The vapors/condensate was then collected in a 2 L cylindrical vessel which was jacketed with wet ice. The 2 L cylinder was evacuated of non-condensables prior to the pressure flash. The vacuum was then broken, and the condensate collected. The same system was then used to collect the vacuum flash condensate.

The second set of equipment consisted of a 2 L jacketed, horizontal glass reactor connected to a hot water recirculation bath. During ammonia treatment experiments, the temperature of the bath was set to 70° C. and vacuum was applied to remove excess NH₃. The glass reactor was further equipped to collect condensate as described above.

### Saccharification Equipment

Saccharification experiments were conducted in stirred tank reactors, where the experiments were done in batch or fed batch mode. The system consisted of a glass jacketed cylindrical reaction vessel, either 500 mL or 2000 mL (LabGlass Number LG-8079C, LabGlass, Vineland, N.J.), equipped with a four neck Reaction Vessel Lid (LG-8073). A stirrer was mounted through the central port to stir the reactor contents. A glass condenser was connected to one of the necks and was kept chilled at 5° C., by recirculating water from a chiller. The other two ports were used for loading of reactants and for temperature and pH measurements. The reactor temperature was controlled by recirculating hot water, supplied by a heated circulator water bath. A four-paddle glass stirrer with 45 degree angled paddles was used as the agitator in the 500 mL reactor. A triple, four-blade stainless steel stirrer was used in the 2-L reactors.

### Fermentation Equipment

Small scale temperature- and pH-controlled fermentations were performed in Wheaton 50 mL double-arm glass CELSTIR® cell culture flasks (VWR #62401-902, VWR, West Chester, Pa.). The top cap was modified by drilling two holes, one to allow insertion of a plastic capillary line for feeding base for pH control and the other for attaching a 0.2 micron sterile filter to allow gas to escape while maintaining sterility in the CELSTIR® flask. One of the side arm caps was also drilled to allow insertion of a 12 mm diameter pH electrode (Cole-Parmer #EW-59001-65, Cole-Parmer, Vernon Hills, Ill.) for continuous pH measurements. The pH was maintained at a set point using a Eutech alpha-pH200 ⅛ DIN pH Controller (Cole-Parmer #EW-56700-00) and by delivering 4N NaOH with a self-priming 10 μL/stroke micro pump (#120SP1210-5TE, Western Analytical Products, Wildomar, Calif.). The flasks were stirred at about 60 rpm using low profile IKA Squid magnetic stirrers (VWR #33994-354). The second capped arm of the CELSTIR® flask was used for access to remove samples during fermentation for analysis. For temperature control the CELSTIR® flasks and supporting equipment were placed in a VWR Signature Incubator (VWR Model 1545, #35823-204).

Fermentation microorganism

The fermentability of the hydrolysates was tested with a stress adapted strain of Zymomonas mobilis designated ZW705, which was itself derived from Z. mobilis strain ZW801-4. The adaptation of ZW801-4 to stress conditions was described in commonly owned Published Patent Application WO 2010/075241, which is herein incorporated by reference. ZW801-4 is a recombinant xylose-utilizing strain of Z. mobilis that was described in commonly owned and co-pending Published Patent Application US 2008/0286870, which also is herein incorporated by reference. Strain ZW801-4 was derived from strain ZW800, which was derived from strain ZW658, all as described in Published Patent Application US 2008/0286870. ZW658 was constructed by integrating two operons, P_{gap}xylAB and P_{gap}taltkt, containing four xylose-utilizing genes encoding xylose isomerase, xylulokinase, transaldolase and transketolase, into the genome of ZW1 (ATCC #31821) via sequential transposition events, and followed by adaptation on selective media containing xylose. ZW658 was deposited as ATCC #PTA-7858. In ZW658, the gene encoding glucose-fructose oxidoreductase was insertionally-inactivated using host-mediated, double-crossover, homologous recombination and spectinomycin resistance as a selectable marker to create ZW800. The spectinomycin resistance marker, which was bounded by loxP sites, was removed by site specific recombination using Cre recombinase to create ZW801-4.

All fermentations were performed in 50 mL reactors (described in Methods) at 33° C. and in medium adjusted to pH 5.8. To allow the following of cell growth, the hydrolysates were clarified by centrifugation (Sorvall SS34 rotor at 45,000×g for 20 minutes) followed by filtration through a sterile 0.2 micron filter unit (Nalgene). The seed culture for inoculating the hydrolysates was grown in a yeast extract medium containing 20 g/L yeast extract, 4 g/L KH₂PO₄, 2 g/L MgSO₄.7H₂O, 1.8 g/L sorbitol, and 150 g/L glucose. The pH was maintained at 5.8 in both the seed culture and the hydrolysates by using 4 N NaOH as a base. The change in OD (600 nm) of the cultures was measured over time correcting for the background absorbance of the medium. Glucose and xylose consumption and ethanol production were monitored by HPLC analysis of removed aliquots.

For best results the seed reactor is typically allowed to reach about 10 OD and then a 10% seed inoculum is added to the hydrolysate reactor. To provide a more challenging test it was decided to add a smaller amount of seed culture. When the OD of seed culture reached 14 (40 g/L glucose remaining), 0.67 mL of seed culture plus 4.33 mL of seed medium was transferred to 45 mL of hydrolysate in each of four reactors.

### Examples 1-3 and Comparative Example A

The following Examples demonstrate the present methods of ammonia treatment of biomass for release of fermentable sugars with an improved inhibitor profile. To quantify the yield of sugars obtained and to demonstrate the benefits of the improved inhibitor profile, the ammonia-treated biomass samples were saccharified and the hydrolysate then subjected to fermentation.

Comparative Example A is included to illustrate the saccharification and fermentation results for biomass pretreated by an alternative ammonia process which produced an inhibitor profile in which the acetamide to acetate ratio was less than one. Fermentation of the hydrolysate obtained by saccharification of the alternatively pretreated biomass demonstrated a lower rate of fermentation under the same fermentation conditions used for Examples 1-3.

### Ammonia Treatment of Example 1

A horizontal cylindrical paddle mixer reactor with a nominal working volume of 5 L was charged at atmospheric pressure and 22° C. with 520 grams of corn cob which had been hammer milled through a 1-mm size screen. The hammer milled corn cob had an initial moisture content of approximately 5 wt % (i.e. 95% dry matter). To enhance contacting with ammonia, and to minimize pressure build-up during reaction, the reactor was evacuated of non-condensables to an absolute pressure of approximately 75 mm Hg before 138 grams of 29 wt % ammonium hydroxide solution was pumped into the reactor. The contents were allowed to mix for 30 minutes and the mixer was then shut off. A recirculation water bath was connected to the reactor jacket with the bath temperature set to 37° C. The resulting ammonia loading was 8 weight percent of dry matter, and the initial solids loading was 76%. The reaction was allowed to proceed for 68 hours at 37° C. Atmospheric steam was then applied to the reactor jacket while sweeping the reactor head space with nitrogen to remove excess ammonia. After 1.5 hours of heating at 100° C., the final product was removed from the reactor.

### Ammonia Treatment of Example 2

A horizontal cylindrical paddle mixer reactor with a nominal working volume of 2 L was charged with 294.1 grams of corn cob which had been hammer milled through a 1-mm sized screen. The hammer milled corn cob had an initial moisture content of approximately 5 wt % (i.e. 95% dry matter). To adjust the initial moisture of the cob to 15 wt %, 35.0 g of room temperature water were added to the cob, and allowed to mix for approximately 30 minutes. Next, 38.3 grams of 29 wt % ammonium hydroxide were added and allowed to mix for 15 minutes. The mixer was then turned off. The resulting ammonia loading was 4 wt % of dry matter, and the initial solids loading was 77 wt %. Water at 37° C. was circulated through the reactor jacket. The reaction was allowed to proceed for 118 hours at 37° C. A vacuum was then applied to reduce the system pressure to approximately 25 mm Hg, and the recirculation batch temperature was increased to 70° C. for 120 minutes in order to remove excess ammonia from the system. The final product was then removed from the reactor.

### Ammonia Treatment of Example 3

This example was done using a procedure similar to Example 2. A horizontal cylindrical paddle mixer reactor with a nominal working volume of 2 L was charged with 350.0 grams of corn cob which had been hammer-milled through a 1-mm sized screen. The hammer milled corn cob had an initial moisture content of approximately 5 wt % (i.e. 95% dry matter). To adjust the initial moisture of the cob to 15 wt %, 42.9 grams of room temperature water were added to the cob, and allowed to mix for approximately 5 minutes. Next, 46.1 grams of 29 wt % ammonium hydroxide were added and allowed to mix for 10 minutes. The mixer was then turned off. The resulting ammonia loading was 4.0 wt % of dry matter, and the initial solids loading was 76 wt %. Water at 37° C. was circulated through the reactor jacket. The reaction was allowed to proceed for 118 hours at 37° C. A vacuum was then applied to reduce the system pressure to approximately 25 mm Hg, and the recirculation batch temperature was increased to 70° C. for 120 minutes in order to remove excess ammonia from the system. The final product was then removed from the reactor.

### Ammonia Treatment of Comparative Example A

Comparative Example A was based on pretreatment experiments conducted using a 130 L nominal working volume horizontal cylindrical pretreatment reactor. A series of ten individual 130 L pretreatment reactor experiments were conducted to produce sufficient pretreated material to conduct a 1000 L saccharification experiment. The hydrolysate from the 1000 L experiment was used for comparing fermentation performance to the hydrolysates generated from pretreatment experiments as described in the above Examples 1 through 3. The description below describes the average conditions used for each of the 130 L pretreatment experiments.

Hammer milled corn cob, which passed through either a ⅜″ or 3/16″ screen, was charged into the reactor. The moisture content of the cob was approximately 8.5 wt %. For each batch, the reactor was charged with 29.7 kg of cob. Ammonium hydroxide and water were charged into the reactor so that the initial ammonia loading was either 6 wt % DM (6 batches) or 8 wt % DM (4 batches). Average ammonia loading for the cobs charged to the saccharification was 6.8 wt % of DM. The initial solid loading was an average of 55.8 wt %. The reactor was preheated using steam on the jacket to a temperature of 75-95° C. Steam was directly injected into the reactor to raise the reaction temperature to approximately 140° C. in a time of approximately four minutes. After reaching the target temperature of greater than 140° C., the reaction mixture was held for 20 minutes at a temperature controlled to 145° C. ±2° C. The pressure in the system was then let down to atmospheric pressure, before vacuum was applied to remove excess aqueous ammonia vapor to a condenser and scrubber system. When the temperature of the reactor was less than about 60° C., the pretreated product was removed from the reactor.

The following table summarizes the ammonia treatment conditions and results for Examples 1-3 and Comparative Example A. Numerical values given for Comparative Example A are averages of the 10 individual pretreatments.

TABLE 2
Pretreatment Conditions and Results for
Examples 1-3 and Comparative Example A

| | | Example | | | |
|---|---|---|---|---|---|
| | | | | | Compar- |
| | Units | 1 | 2 | 3 | ative |
| NH₃ Loading | wt % of DM | 8.00 | 4.00 | 4.00 | 6.76 |
| Feedstock Solids | % DM | 95.0 | 85.0 | 85.0 | 91.5 |
| Initial Solids | wt % of | 76.0 | 77.0 | 76.0 | 55.8 |
| Loading | total charge | | | | |
| Residence Time | hrs | 68 | 118 | 118 | 0.33 |
| Reaction | ° C. | 37 | 37 | 37 | 143 |
| Temperature | | | | | |
| Residual NH₃ | wt % of DM | 0.16 | 0.24 | 0.20 | 0.28 |
| Acetic Acid | mole percent | 12.6 | 24.5 | 21.6 | 48.7 |
| Conversion | | | | | |
| Acetamide | mole percent | 78.5 | 74.1 | 70.4 | 49.5 |
| Conversion | | | | | |
| Total Acetyl | mole percent | 91.1 | 98.5 | 92.0 | 98.2 |
| Conversion | | | | | |
| AM/AA ratio | mole/mole | 6.2 | 3.0 | 3.3 | 1.0 |

### Saccharification of the Ammonia-Treated Biomass of Examples 1, 2, and 3

The ammonia-treated cobs of Examples 1, 2, and 3 were saccharified separately in 0.5 L reactors. In these runs, the biomass was charged in fed-batch mode, while the enzymes were charged in batch mode, at the beginning of the experiments. The ammonia-treated cobs were saccharified without further size reduction. De-ionized water was used as the reaction heel. Ammonia-treated cobs were added to the water to make slurries of about 12.5% DWB. The temperature was increased to 47 C and the pH was adjusted to 5.3 using a 1N sulfuric acid solution. The enzymes were added in the following doses based on final hydrolysate: SPEZYME® CP and Novozyme-188 at 20 and 5 mg protein/g of cellulose, respectively, and MULTIFECT®-CX12L at 10 mg protein per gram of hemicellulose. The remaining pretreated cobs were charged in three equal portions within 4 hours after addition of enzymes to bring the total solids loading of the hydrolysate to 25% DWB. The reactors were continuously stirred at 300-500 rpm to maintain the particles suspended and well stirred throughout the run. After 72 hours, the sugar content of the resulting saccharification liquor was measured according to the sugar measurement protocol described in the General Methods. The saccharification results are shown in Table 3 as percent of theoretical yield.

### Saccharification of Comparative Example A Pretreated Biomass

Saccharification of Comparative Example A biomass, which had been pretreated as described above, was performed in a 1450 L reactor containing about 100 L hydrolysate. The enzymes and their dosages were identical to those in Examples 1, 2, and 3. The charging of biomass was similar to those of the Examples 1, 2, and 3, except that after the initial loading and addition of enzymes, the remaining biomass was added continuously in nine hours. The main difference of Comparative Example A with Examples 1, 2, and 3 was the utilization of a recirculation loop with an in-line grinder in the reactor used in Comparative Example A. The in-line grinder reduced the particle size distribution of the biomass during the run, increasing the saccharification rates and increasing the yields of sugar formation.

TABLE 3
Yields of Sugars in the Liquid Phase Through Saccharification
for Examples 1-3 and Comparative Example A.

| | monomer | oligomer | | total | monomer | oligomer | total |
|---|---|---|---|---|---|---|---|
| Example | glucose | glucose | cellobiose | glucose | xylose | xylose | xylose |
| 1 * | 41.9 | 10.5 | 3.0 | 55.4 | 33.3 | 41.8 | 75.1 |
| 2 * | 35.5 | 12.6 | 2.4 | 50.5 | 23.9 | 44.4 | 68.3 |
| 3 * | 40.0 | 7.7 | 2.7 | 50.4 | 31.7 | 42.6 | 74.3 |
| Comparative | 45.57 | 11.39 | 6.92 | 63.88 | 36.24 | 52.56 | 88.80 |
| Example A ** | | | | | | | |

* Yields at 72 hours
** Yields at 70 hours

The molar ratio of acetamide to acetic acid was 1.13 at 70 hours, which remained essentially constant throughout saccharification, varying from 1.13 to 1.17. The acetamide to acetic acid ratio of 1.13 for Comparative Example A compares with 4.98, 2.78, and 2.62 in Examples 1, 2, and 3, respectively.

The glucose and xylose yields of Comparative Example A are somewhat higher than those of Examples 1, 2, and 3, mainly because Comparative Example A used an in-line grinder during saccharification. The in-line grinder reduced the particle size distribution of the biomass, increasing the rates of sugar formation. Xylose formation in all cases was similar. Most of the total xylose is formed in the first 24 hours, followed by a slow increase during the remaining duration of the run.

### Fermentation of Hydrolysates of Examples 1-3 and of Comparative Example A

Fermentation performance of Zymomonas mobilis strain ZW705 was used to evaluate corn cob hydrolysates of Examples 1-3 relative to that of Comparative Example A in a side-by-side manner beginning with identical seed cultures for each fermentation. Strain ZW705 is a recombinant strain containing integrated transgenes that allow Zymomonas to ferment xylose as well as glucose. The generation of this strain is described above and has been described in commonly owned and co-pending U.S. Patent Application No. 61/139,852 filed Dec. 22, 2009.

All fermentations were performed in 50 mL reactors (described above) at 33° C. and in media adjusted to pH 5.8. To allow the following of cell growth, the hydrolysates were clarified by centrifugation (Sorvall SS34 rotor at 45,000×g for 20 minutes) followed by filtration through a sterile 0.2 micron filter unit (Nalgene). The seed culture for inoculating the hydrolysates was grown in a yeast extract medium containing 20 g/L yeast extract, 4 g/L KH₂PO₄, 2 g/L MgSO₄.7H₂O, 1.8 g/L sorbitol, and 150 g/L glucose. The pH was maintained at 5.8 in both the seed culture and the hydrolysates by using 4 N NaOH as a base. The change in optical density (at 600 nm) of the cultures was measured over time correcting for the background absorbance of the medium. Glucose and xylose consumption and ethanol production were monitored by HPLC analysis of removed aliquots.

For best results the seed reactor is typically allowed to reach about 10 OD and then a 10% seed inoculum is added to the hydrolysate reactor. To provide a more challenging test it was decided to add a smaller amount of seed culture. When the OD of seed culture reached 14 (40 g/L glucose remaining), 0.67 mL of seed culture (1.3% by volume) plus 4.33 mL of seed medium was transferred to 45 mL of hydrolysate in each of four reactors. Data from the resulting fermentations are shown below in the Table.

From the data, it can be seen that the fermentations of the hydrolysates of Examples 1-3 show a shorter lag and faster growth rate than the fermentation of the hydrolysate of Comparative Example A. The total fermentation time is also much faster, even when accounting for the ˜25% more total sugar in Comparative Example A hydrolysate. The Table below lists the volumetric fermentation rates, titer and yields. While the yields for all four fermentations were about the same, the maximum glucose and xylose uptake rates, the average ethanol production rate, and the maximum growth rate were all faster with the hydrolysates of Examples 1-3 compared with the hydrolysate of Comparative Example A. Only the titer was higher in Comparative Example A due to its higher starting total sugar content.

TABLE 4
Data from Fermentations.

| | Ex. 1 | Ex. 2 | Ex. 3 | Comp. Ex. A |
|---|---|---|---|---|
| Max GUR (g/L/h) | 7.4 | 7.2 | 6.8 | 5.2 |
| Max XUR (g/L/h) | 3.8 | 4.0 | 3.1 | 2.7 |
| Max EtOH Titer (g/L) | 45.0 * | 47.6 * | 42.0 ** | 58.7 *** |
| Avg EtOH Rate (g/L/h) | 1.6 * | 1.7 * | 1.7 ** | 1.0 *** |
| % Yield EtOH | 89 * | 88 * | 89 ** | 89 *** |
| Max Growth Rate (h⁻¹) | 0.25 | 0.22 | 0.24 | 0.13 |
| Initial Growth Lag (h) | 2.8 | 2.7 | 1.5 | 6.1 |
| Initial Glucose (g/L) | 63.5 | 65.8 | 57.8 | 78.1 |
| Final Glucose (g/L) | 0.0 | 0.0 | 0.0 | 0.0 |
| Initial Xylose (g/L) | 31.4 | 36.7 | 31.4 | 46.3 |
| Final Xylose (g/L) | 1.1 | 1.1 | 1.1 | 4.0 |

Notes:
* value at 27 h
** value at 24 h
*** value at 54 h

### Examples 4-11 and Comparative Examples B-G

The following Examples demonstrate the present methods of ammonia treatment of biomass for release of fermentable sugars with an improved inhibitor profile. Comparative Examples B through G are included for comparison purposes.

Anhydrous ammonia (4.0 grade, lecture bottle 2″×13″ size) was obtained commercially from GT&S Inc. (Allentown, Pa.). Corn cob obtained from University of Wisconsin Farm, in Madison, Wis. and having a composition similar to that indicated in Table 1 was hammermilled to 1.0 mm by treating in a micropulverizer (Model #1SH, Serial #10019; Pulverizing Machinery Division of Mikropul Corporation; Summit, N.J.) with a 1.0 mm screen. Dry ice was added to the cob before grinding to prevent overheating of equipment. Dry matter content of biomass was determined using a Denver Instruments IR-120 moisture analyzer operating at 105° C. The measurement method for acetamide and acetic acid was similar to that described herein above except that the column was operated at 65° C. instead of 55° C.

The ammonia treatment system used for Examples 4-11 and Comparative Examples B-G consisted of a 75 mL stainless steel high pressure tube (Hoke, Inc., Spartanburg, S.C.) modified to include a Cole Parmer pressure transducer (Model 206) on one end. This end of the tube was connected to a coiled line which was connected to a vacuum line and to the anhydrous ammonia source. The other end of the tube was used as a port for adding the cob by use of a funnel. A small amount of cob, equivalent to 14% of the tube's volume capacity, was uniformly distributed on the bottom of the tube; the cob was loosely packed inside the tube so that the ammonia could interact uniformly with the cob. After cob addition, a thermocouple was put through the port and the tube was closed. The tube and the coil line were immersed into a water bath set at a given temperature for temperature control. The thermocouple and pressure transducer were connected to a data acquisition box and wired to a laptop with DaqView software (Measurement Computing Corp., Norton, Mass.) for electronic acquisition of pressure and temperature readings inside the tube. A scrubber containing 37% HCl was connected upstream of the vacuum pump to neutralize any ammonia vented out of the tube. A needle valve was used to slowly add the desired amount of ammonia for each experiment. The amount of ammonia added was measured by placing the ammonia lecture bottle on an electronic balance and recording the weight before and after addition of ammonia into the tube.

The following pretreatment procedure was used. A horizontal pressure tube with nominal volume of 75 mL was charged at atmospheric pressure with 3.6 to 3.8 g of cob having a percent moisture as indicated in Table 5. This moisture in the cob was the source of water in the experiments. To reach a desired percent moisture, water was added to hammermilled cob having an initial moisture content of approximately 4.5% (i.e. 95.5% dry matter). The cob mixture was then stirred for at least minutes using a spatula and placed in a refrigerator overnight to reach equilibrium. Next day, the cob was mixed again for 5 minutes and a sample of this mixture was analyzed to determine the percent moisture content.

After adding the cob, the tube was sealed and placed in a water bath until the desired temperature was reached inside the tube. To enhance contacting with ammonia, the immersed tube was evacuated to an absolute pressure of 0.1 bara before addition of anhydrous ammonia. For pretreatment experiments done at 70° C., the ammonia was allowed to remain in the tube with the cob for 15 min, at which point the tube was transferred to an iced water bath to lower its temperature. Vacuum was then applied to reach 0.1 bara to remove excess ammonia. Nitrogen was applied to bring the tube pressure back to atmospheric pressure before removing the tube from the water bath and removing the product for analysis.

Table 5 summarizes the reaction conditions used for Examples 4-11 and Comparative Examples B-G, and the results obtained.

TABLE 5
Pretreatment Conditions and Results for Examples 4-11 and Comparative Examples B-G.

| | Feedstock | NH3 loading | Initial solids loading | Ratio water | Total acetyl | AM/AA |
|---|---|---|---|---|---|---|
| | solid | wt % of | wt % of total | to NH3 | conversion | ratio |
| Sample | (% moisture) | DM | charge | g/g | mole % | mole/mole |
| Ex. 4 | 36.3 | 6.4 | 61.2 | 8.9 | 60.6 | 2.1 |
| Comp. Ex. B | 36.3 | 3.6 | 62.3 | 15.9 | 45.5 | 1.0 |
| Ex. 5 | 36.3 | 11.2 | 59.5 | 5.1 | 67.0 | 2.5 |
| Comp. Ex. C | 36.3 | 2.1 | 62.9 | 27.3 | 17.3 | 0.4 |
| Ex. 6 | 28.2 | 5.6 | 69.0 | 6.9 | 69.1 | 2.3 |
| Ex. 7 | 28.2 | 5.6 | 69.1 | 7.0 | 72.1 | 2.1 |
| Comp. Ex. D | 28.2 | 3.8 | 69.9 | 10.3 | 51.9 | 1.3 |
| Comp. Ex. E | 28.2 | 2.4 | 70.6 | 16.3 | 28.0 | 0.6 |
| Ex. 8 | 28.2 | 9.9 | 67.1 | 4.0 | 78.2 | 2.9 |
| Ex. 9 | 81.9 | 6.5 | 77.7 | 3.4 | 66.6 | 2.7 |
| Ex. 10 | 81.9 | 9.6 | 75.9 | 2.3 | 77.5 | 3.7 |
| Comp. Ex. F | 81.9 | 3.5 | 79.6 | 6.2 | 28.3 | 1.2 |
| Ex. 11 | 90.6 | 9.5 | 83.4 | 1.1 | 66.1 | 3.4 |
| Comp. Ex. G | 90.6 | 7.6 | 84.7 | 1.4 | 57.9 | 3.1 |

All runs were performed in pressure tubes at 70° C. with a 15 minute pretreatment time.

The results in Table 5 show that for a given pretreatment time and temperature, the percent ratio of biomass, water and ammonia can be adjusted to reach optimal product specifications with respect to total acetyl conversion and AM/AA ratio.

Although particular embodiments of the present invention have been described in the foregoing description, it will be understood by those skilled in the art that the invention is capable of numerous modifications, substitutions, and rearrangements without departing from the spirit of essential attributes of the invention. Reference should be made to the appended claims, rather than to the foregoing specification, as indicating the scope of the invention.

What is claimed is:

1. A method for treating biomass for release of fermentable sugars with an improved inhibitor profile, the method comprising:
a) treating biomass with an amount of ammonia under suitable reaction conditions wherein said conditions provide for a pretreated biomass reaction product having an acetamide to acetate molar ratio greater than about 1 and an acetyl conversion of greater than 60%, wherein said suitable reaction conditions include pressure from about sub-atmospheric pressure to less than 10 atmospheres;
b) saccharifying the pretreated biomass reaction product with at least one saccharification enzyme, wherein a hydrolysate comprising fermentable sugars is produced and wherein said hydrolysate has an improved inhibitor profile compared to saccharifying a pretreated biomass reaction product having an acetamide to acetate molar ratio of less than about 1; and
c) maintaining the acetamide to acetate molar ratio greater than about 1 throughout the saccharifying of step (b).

2. The method of claim 1, further comprising fermenting the hydrolysate to produce a target product by adding an inoculum of seed cells capable of fermenting sugars to a target product.

3. A method for fermenting sugars to a target product, the method comprising:
a) providing a hydrolysate of claim 1 having an acetamide to acetate molar ratio greater than about 1;
b) adding an inoculum of seed cells capable of fermenting sugars to a target product to said hydrolysate, wherein the inoculum is about 0.1 percent to about 10 percent of the hydrolysate; and
c) fermenting the hydrolysate to provide a fermentation mixture comprising a target product.

4. The method of claim 2 or 3, wherein the hydrolysate provides for improved cell growth rate of said inoculum compared to a hydrolysate having an acetamide to acetate molar ratio of less than 1.

5. The method of claim 2 or 3, wherein fermenting the hydrolysate is initiated with a lower inoculum of seed cells compared to fermenting a hydrolysate having an acetamide to acetate molar ratio of less than 1.

6. The method of claim 1, wherein the acetyl conversion is greater than 70%.

7. The method of claim 1, wherein total xylose yield through saccharification is improved compared to that for a pretreated biomass reaction product having an acetamide to acetate molar ratio of less than about 1 and an acetyl conversion of greater than 60%.

8. The method of claim 1, wherein the biomass has a dry matter content of at least about 60 weight percent in step (a).

9. The method of claim 1, wherein the suitable reaction conditions include a mass ratio of water to ammonia of less than about 20:1.

10. The method of claim 1, wherein the suitable reaction conditions include a system solids loading of greater than about 60%.

11. The method of claim 1, wherein the biomass is subjected to preprocessing prior to step (a).

12. The method of claim 1, wherein the suitable reaction conditions include a temperature of about 4° C. to about 200° C. and a reaction time of 30 days or less.

13. The method of claim 13, wherein the temperature is about 20° C. to about 121° C. and the reaction time is about 100 hours or less.

14. The method of claim 2 or 3, wherein the target product is selected from the group consisting of ethanol, butanol, and 1,3-propanediol.
